(19) United States
(12) Patent Application Publication
(10) Pub. No.: US 2017/0151388 A1
(43) Pub. Date: Jun. 1, 2017

(54) Motorized Drug Delivery Device

(71) Applicant: Novo Nordisk A/S, Bagsvaerd (DK)

(72) Inventors: Michael Monrad, Frederiksberg (DK); Per Einar Pontus Holm, Tygelsjoe (SE); Jens Aage Munk, Oelstykke (DK)

(73) Assignees: Novo Nordisk A/S, Bagsvaerd (DK); Novo Nordisk A/S, Bagsvaerd (DK)

(21) Appl. No.: 15/324,559

(22) PCT Filed: Jul. 8, 2015
(86) PCT No.: PCT/EP2015/065544

§ 371 Date: Jan. 6, 2017

(30) Foreign Application Priority Data
Jul. 9, 2014 (EP) 14176294.8

(51) Int. Cl. A61M 5/20; A61M 5/315

(52) U.S. Cl. CPC A61M 5/20; A61M 5/31546; A61M 5/31578; A61M 2005/31588; A61M 2205/3368; A61M 2005/3126

(57) ABSTRACT

A drug delivery device comprising a piston rod adapted to engage and axially move a cartridge piston to thereby expel an amount of drug from a loaded cartridge corresponding to a set dose amount, the device comprising a motor for moving the drive member from an initial position, through a start-of-dose position, to an end-of-dose position, as well as position detection means adapted to generate output data indicative of the drive member's position relative to the piston of a loaded cartridge. An electronic controller adapted is adapted operate the motor to continuously move the drive member from the initial position, based on output data from the position detection means determine that the drive member is in engagement with the cartridge piston corresponding to the start-of-dose position, and operate the motor to move the drive member from the start-of-dose position to the an end-of-dose position corresponding to the set dose.

The present invention generally relates to drug delivery devices adapted to be used and operated by a patient on his or her own hand. More specifically the invention relates to motorized drug delivery devices.

### BACKGROUND OF THE INVENTION

In the disclosure of the present invention reference is mostly made to the treatment of diabetes by subcutaneous drug delivery, either discrete or continuous, however, this is only an exemplary use of the present invention.

The most common type of durable drug delivery devices adapted to receive a drug filled cartridge and expel a discrete dose of a desired size therefrom are driven by manual means or by a spring energized during dose setting, the cartridge being of the type comprising an axially displaceable piston having an initial proximal position and which is moved distally by a piston rod. Subcutaneous drug delivery takes place via an injection needle arranged in fluid communication with the cartridge. The device may be pen-formed or in the form of a more box-shaped so-called doser. In order to improve convenience, user-friendliness and provide additional features, e.g. detection and storing of expelling data, drug delivery devices have been provided with electrically driven means, typically in the form of an electronically controlled motor driving a piston rod through a gear arrangement, e.g. as shown in U.S. Pat. No. 6,514,230, WO 2010/089310 and US 2011/306927.

Whereas motorized drug delivery devices for treatment of diabetes by discrete injections of e.g. insulin are used relatively rarely, in the field of continuous drug delivery motorized drug delivery devises have been used widely for decades. The latter type of devices are generally known as infusion pumps and are normally engineered to very high standards and are correspondingly very expensive.

For example, WO 2007/094833 discloses a drug delivery system which is adapted to mitigate the effects of differential thermal expansion or contraction between a fluid and a container for controlled delivery of the fluid.

Although a motorized drug delivery device for discrete injections of drug also has to meet very high safety standards, the cost issue is more important as the relatively inexpensive mechanical drug delivery devices, e.g. of the pen-type, to most users are an acceptable alternative. Correspondingly, to make the higher expense acceptable to the user additional advantages should be offered.

Having regard to the above, it is an object of the present invention to provide a motorized drug delivery device which provides a high degree of user-friendliness in a reliable, robust and cost-effective way.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in accordance with a first general aspect of the invention a drug delivery device is provided comprising a drug-filled cartridge or means for receiving a drug-filled cartridge in a loaded position, the cartridge comprising an outlet and an axially displaceable piston. The device further comprises means for setting a dose corresponding to a desired dose amount, a drive member adapted to engage and axially move the piston to thereby expel an amount of drug from a loaded cartridge through the outlet corresponding to the set dose amount, and a motor for moving the drive member from (i) an initial position, through (ii) a start-of-dose position, to (iii) an end-of-dose position. The device also comprises position detection means adapted to generate output data indicative of the drive member's position relative to the piston of a loaded cartridge, and an electronic controller adapted to (i) operate the motor to move the drive member from the initial position, (ii) based on output data from the position detection means determine that the drive member is in engagement with the cartridge piston corresponding to the start-of-dose position, and (iii) operate the motor to move the drive member from the start-of-dose position to the an end-of-dose position corresponding to the set dose. The motor is operated continuously as the drive member is moved from the initial to the end-of-dose position.

By this arrangement an air gap between the drive member and cartridge piston can be eliminated automatically “on- the-fly” without user involvement.

In an exemplary embodiment the electronic controller is adapted to determine the start-of-dose position based on a time-shifted calculation, this allowing the processor to determine the “true” start-of-dose position more precisely, this resulting in a more precise and reliable dosing. Such an arrangement addresses the issue that a given position detection means will not provide data which allows the controller to determine in real-time that the drive member has just engaged the cartridge piston.

For example, the start-of-dose position may be determined after the drive member has engaged the cartridge piston, e.g. by a force sensor arranged at least partly in the drive member, this allowing the controller to filter and analyse data received after the drive member has engaged the cartridge piston. Indeed, for any given system the determination should be sufficiently fast to take place before the piston driver has moved a distance corresponding to the expelling of a set dose.

Alternatively, the start-of-dose position may be determined before the drive member engages the cartridge piston, e.g. the position detection means may comprise a proximity sensor arranged at least partly in the drive member.

The motor may be operated to move the drive member proximally a distance after the drive member has reached the end-of-dose position, e.g. immediately thereafter to reduce after drip. Alternatively the motor may be operated to move the drive member proximally a distance from the initial position before the drive member is moved continuously from the initial to the end-of-dose position.

The motor may be operated at different speeds during the different periods of movement. For example, the piston rod may be moved from its proximal-most position towards the piston at a first speed which is then lowered as the piston rod approaches the piston, which may be used to optimize precise detection of the start-of-dose position. Correspondingly, during the expelling stroke the speed may be lowered as the piston rod approaches the end-of-dose position.

In accordance with a further aspect of the invention a method of operating a drug delivery device is provided. The method comprises the steps of (i) providing a drug delivery device comprising a drug-filled cartridge comprising an outlet and an axially displaceable piston, drug expelling means comprising means for setting a dose corresponding to a desired dose amount, a drive member adapted to engage and axially move the piston to thereby expel an amount of drug from a loaded cartridge through the outlet corresponding to the set dose amount, and a motor for moving the drive member from (i) an initial position, through (ii) a start-of-dose position, to (iii) an end-of-dose position, as well as position detection means adapted to generate output data indicative of the drive member's position relative to the piston of a loaded cartridge, and an electronic controller. The method comprises the further steps of (ii) operating the motor to move the drive member from the initial position, (iii) based on output data from the position detection means determining that the drive member is in engagement with the cartridge piston corresponding to the start-of-dose position, and (iv) operating the motor to move the drive member from the start-of-dose position to the an end-of-dose position corresponding to the set dose, wherein the motor is operated continuously as the drive member is moved from the initial to the end-of-dose position.

The step of determining the start-of-dose position may be based on a time-shifted calculation, e.g. the start-of-dose position may be determined after the drive member has engaged the cartridge piston, or the start-of-dose position may be determined before the drive member has engaged the cartridge piston.

In accordance with a second general aspect of the invention a drug delivery device is provided comprising a drug-filled cartridge or means for receiving a drug-filled cartridge, the cartridge comprising an outlet and an axially displaceable piston. The drug delivery device further comprises drug expelling means comprising a drive member adapted to engage and axially move the piston to thereby expel an amount of drug from the cartridge through the outlet, and a motor for moving the drive member, and an electronic controller. The electronic controller is adapted to control the motor to move the drive member in (i) a distal direction to thereby expel an amount of drug, and (ii) a proximal direction to thereby allow the content of a drug-filled cartridge to expand.

By this arrangement the situation in which a drug delivery device with a loaded cartridge is placed in a refrigerator in close proximity to the evaporator or the cold air outlet is addressed, the situation resulting in a risk of the drug freezing. The drug thereby expands and the piston rod arranged in contact with the cartridge piston is pushed back with a high force which may seriously harm the device drive mechanism.

In an exemplary embodiment the drug delivery device comprises a sensor adapted to measure a proximally directed force acting on the piston drive member when the motor is not operated, wherein the controller, based on data from the sensor, is adapted to move the drive member proximally when a given level of force is detected. In this way the drive mechanism can be protected from damage. The distance the drive member is moved in the proximal direction may be correlated to the amount of drug in the cartridge.

In a further exemplary embodiment the drug delivery device comprises a sensor adapted to measure a temperature indicative of the temperature in the cartridge, wherein the controller, based on data from the sensor, is adapted to move the drive member proximally when a given temperature is measured. The distance the drive member is moved in the proximal direction may be correlated to the amount of drug in the cartridge.

In a yet further exemplary embodiment the motor of the drug delivery device, after an amount of drug has been expelled, is controlled to move the drive member in the proximal direction into a “protected state” in which the expelling mechanism cannot be damaged by a proximally-moving piston. The distance the drive member is moved in the proximal direction may be correlated to the amount of drug in the cartridge.

As used herein, the term “drug” is meant to encompass any drug-containing flowable medicine capable of being passed through a delivery means such as a cannula or hollow needle in a controlled manner, such as a liquid, solution, gel or fine suspension. Representative drugs include pharmaceuticals such as peptides (e.g. insulins, insulin containing drugs, GLP-1 containing drugs as well as derivates thereof), proteins, and hormones, biologically derived or active agents, hormonal and gene based agents, nutritional formulas and other substances in both solid (dispensed) or liquid form. In the description of the exemplary embodiments reference will be made to the use of insulin containing drugs. Correspondingly, the term “subcutaneous” infusion is meant to encompass any method of transcutaneous delivery to a subject.

### BRIEF DESCRIPTION OF THE DRAWINGS

In the following exemplary embodiments of the invention will be described with reference to the drawings, wherein

FIG. 1 shows schematically an embodiment of a drug delivery device,

FIG. 2 shows schematically a drive arrangement for a motorized drug delivery device,

FIG. 3 shows data from a first test set-up using a force sensor,

FIG. 4 shows schematically components of an optical drug delivery sensor system,

FIG. 5 shows data from a second test set-up using an optical sensor,

FIG. 6 shows a first embodiment of a drug delivery device platform, and

FIGS. 7 and 8 show a second embodiment of a drug delivery device platform.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

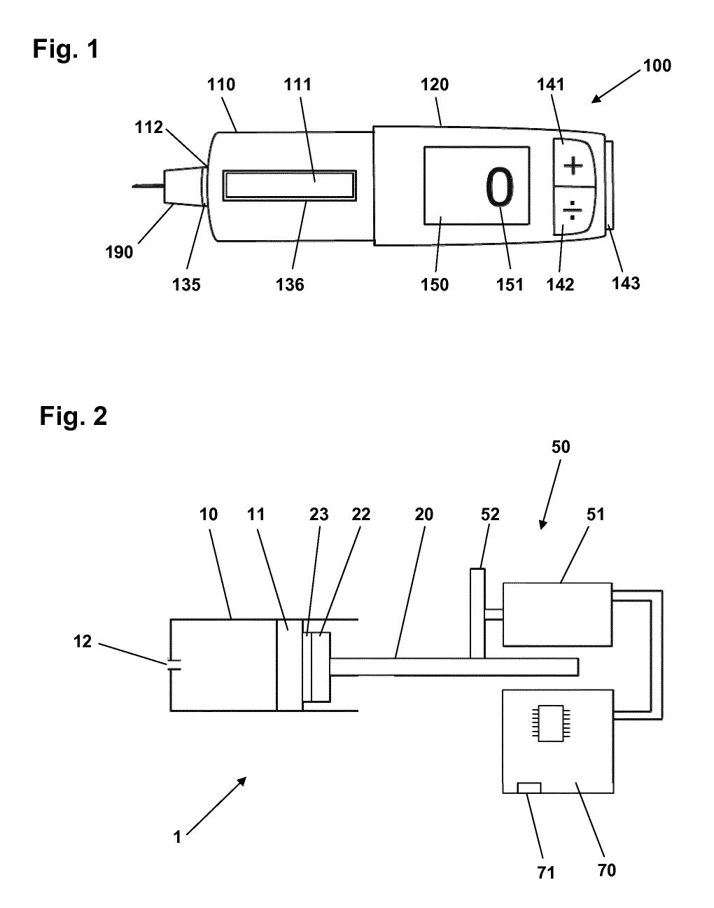

FIG. 1 shows in a schematic representation a generic motorized drug delivery device 100 comprising a main portion 120 in which an expelling assembly is arranged, and a cartridge holder portion 110 adapted to receive and hold an exchangeable drug-filled cartridge. The cartridge holder portion comprises a distal opening 112 and a window 111 allowing a user to visually inspect the content of a loaded cartridge just as the actual position of the cartridge piston can be observed. A needle assembly 190 is mounted in fluid communication with a loaded cartridge. The main portion comprises user input means in the form of a pair of dose setting buttons 141, 142 allowing a user to set and adjust a dose of drug to be expelled, as well as a dose release button 143 arranged at the proximal end of the device. A display 150 shows the currently set dose 151. The display may be controlled to provide further information to a user, e.g. the dose numeral may count down during dose expelling, just as the display may comprises indicators for e.g. battery condition, error conditions, and time.

FIG. 2 shows schematically a drive arrangement for a motorized drug delivery device of the type described with reference to FIG. 1, the arrangement providing a platform for realizing aspects of the present invention.

More specifically, the motorized drug delivery device 1 comprises a main portion in which an expelling assembly 50 is arranged, and a cartridge holder portion adapted to receive and hold an exchangeable drug-filled cartridge 10, the cartridge comprising an axially displaceable piston 11 and a distal outlet 12 associated with coupling means allowing a needle assembly to be mounted. In the shown embodiment the expelling assembly comprises a piston rod 20 adapted to engage and move forward a cartridge piston to thereby expel an amount of drug, the piston rod being driven by an electronically controlled motor 51 via a gear assembly 52. The piston rod comprises a distal piston rod washer 22 in which a sensor 23, e.g. a force sensor, is arranged. The device further comprises electronic controller circuitry 70 adapted to control operation of the motor in order to move the piston rod in a distal or proximal direction, as well as a rechargeable power source (“battery”) associated with the controller circuitry. In the shown embodiment a combined power and data communication port is provided, e.g. a USB port. The controller circuitry is further adapted to receive input from user input means (see below) as well as from one or more sensors, e.g. the shown piston rod sensor, just as the controller circuitry is adapted to control a display in accordance with detected operational conditions. The piston rod sensor may be used to detect piston rod engagement with the cartridge piston when the piston rod is forwarded after cartridge exchange, however, as will be described below, the piston rod sensor may also be used in embodiments of the present invention.

Turning to embodiments of the present invention, the above-described drive arrangement for a motorized drug delivery device is adapted to move the piston from a proximal non-engaged “air gap position” through a piston-engaging position to an end-of-dose position in a single continuous motion. In other words piston-engagement is detected “on the fly” without halting piston rod movement.

This arrangement can provide a number of advantages. For example, during cartridge change the piston rod is typically retracted to a proximal-most position and subsequently has to be advanced to engage the piston of the newly inserted cartridge to remove the air gap. If the motorized drug delivery is manual the user would have to advance the piston rod by performing one or more “air shots” until drug would show from the tip of a mounted needle. In case air gap elimination is automated the drug delivery would move the piston rod distally until engagement with the cartridge piston is detected and then stop movement. However, due to e.g. inertia it is normally not possible to stop the piston rod instantaneously, this resulting in spillage from a mounted needle or pressurization of the cartridge if no needle is mounted, the latter resulting in subsequent spillage when a needle is mounted. Performing air gap removal on-the-fly would remove the above-mentioned issues and improve user-friendliness.

When expelling a given amount of drug using a cartridge-based drug delivery it is recommended to wait e.g. 6-10 seconds before withdrawing the inserted needle, this allowing the elastically deformed system to relax (especially the rubber piston) and the corresponding small amount of drug to be expelled through the inserted needle. Otherwise the user would experience “after dripping” from the needle. To minimize or remove this waiting time a motorized drug delivery device may be adapted to retract the piston rod immediately after having reached the end-of-dose-position, this de-pressurizing the system by allowing the elastically deformed piston to move proximally. As appears, this would require an air gap to be eliminated before each expelling action. Doing this automatically on-the-fly would improve user-friendliness. However, if the piston rod is retracted immediately after having reached the end-of-dose-position, the piston rod should move slightly beyond the calculated end-of-dose position as otherwise under-dosing may occur.

If a drug delivery device after use is stored with a mounted needle drug may seep from the needle, e.g. due to gravity forces or temperature variations, which may result in the cartridge piston moving distally, thereby developing an air gap between the piston and the piston rod. To cope with this situation it is in general recommended to perform an air shot before each injection. Performing this automatically on-the-fly would improve user-friendliness.

In order to perform the above-described continuous distal movement of the piston rod, it is necessary for the controller to precisely detect piston engagement, this defining the axial position from which the dose stroke will begin.

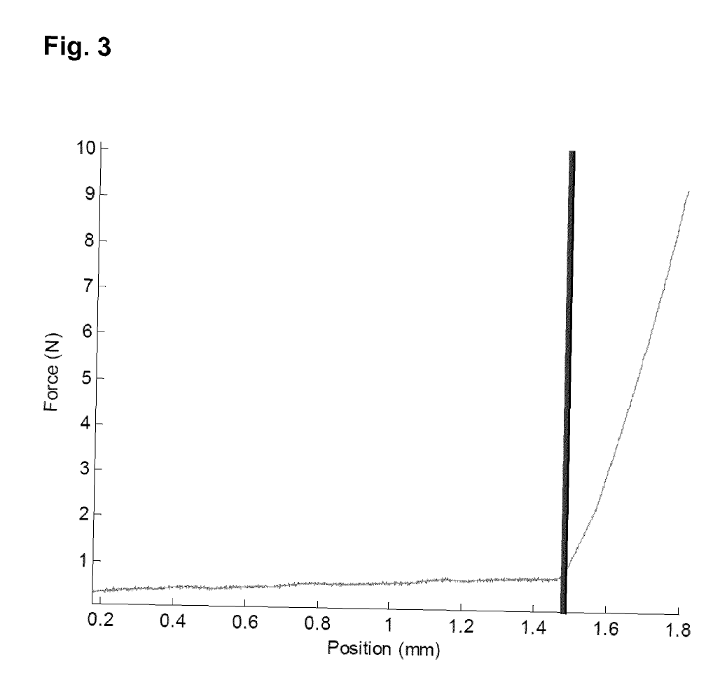

In a first exemplary embodiment engagement is detected using a force sensor arranged at the distal end of the piston rod, i.e. in the piston head. Referring to FIG. 3 data from a test set-up using a 3 mL Novo Nordisk Penfill® cartridge is shown. The figure shows readings from the force sensor as the sensor is moved linearly, contact with the piston taking place at position 1.488 mm. The curve shows the raw force signal from the sensor and a filtered signal combined. As can be seen they are very similar. The black bar is the expected plunger contact and the grey bar is the result of the plunger detection algorithm. In the shown embodiment the algorithm works by differentiating the force signal twice and looks for a peak in the double differentiated signal. Filtering by a FIR (Finite Impulse Response) filter is carried out on the force signal and the differentiated signal. It is noted that there is a good match between the expected plunger contact and the estimated plunger contact. Due to noise results were found to vary, however it appears that a motorized drug delivery device with on-the-fly piston engagement detection can be provided using a distally-mounted piston rod force sensor.

As indicated above, for filtering a FIR filter was chosen. The main reason for the choice was that the phase shift of a FIR filter can be determined. Since the filter will introduce a delay (i.e. the “knee” will advance in time) and this application needs to know the time point of the “knee” this is a crucial design factor.

The filter should be designed as a low pass filter, thus allowing DC and lower frequency signal to pass only. The ripple in pass band is not considered that important, so some ripple can be allowed. The damping of higher frequency signal should be “large”. A sharp cut between the passed and filtered signals is not important either.

A simple moving average approach was chosen and with the amount of tabs (delay elements) in the filter set to 20-30 the filter showed a good filtering while not distorting the signal.

$$y [ nT ] = {\sum }_{m = 0}^{K - 1} h [ mT ] x [ ( n - m ) T ]$$

where T is the sample spacing (in our case 1 ms), K is the number of tabs (delay elements) 20 in our case. h is scaling factor on each delayed signal component and set to 1/K as follows by a moving average. So in other words each output value is the average of the last K input signals. For calculating the result y at initialization the first value of x is “copied” for all m slots. Thus, for m=20, n=0: x[0]=x[−1]=x[−2] . . . =x[−19].

The system in the above-mentioned test set-up runs with a speed of either 5U/s or 10U/s and the sample rate of the force sensor is 1 kHz. The delay of the filter in expelled units will be:

$$a = \frac{{d}_{f}}{sr} \cdot v$$

where a is the amount being expelled during the delay, d_{f} is the number of samples that the filter delays, sr is the sample rate (samples per second) v is the dosing speed (units per second). In the present case of 20 samples of delay the delay of the filter in expelled units will be 0.2 U for a speed of 10 U/s.

Since the differentiated signal is also filtered the total delay of the two times differentiated signal will be the sum of the delays in both filters. Currently that filter has ˜20 tabs, i.e. a filter delay of 0.2U. Thus, the total delay of the filters in expelled units is: 0.2 U+0.2 U=0.4 U. As can be seen in the formula if the speed is 5 U/s the total delay of filters is 0.2 U.

In conclusion, when a FIR filter is used, the delay is known and the filtered signal can be shifted in time to exactly compensate for this delay for an “on the fly” strategy.

Other methods could be used to find the “bend” in the signal. For example, a method could comprise the steps of (i) fitting a line to the first n samples to find the line before the “bend”, (ii) iteratively looking at distance between the fitted line and the samples; when the curve goes upward in the “bend” the distance from the fitted line to the samples will increase, this giving a rough estimate of the “bend”, and (iii) fitting a line before and after the “bend”, the result would be the intersection of the two fitted lines.

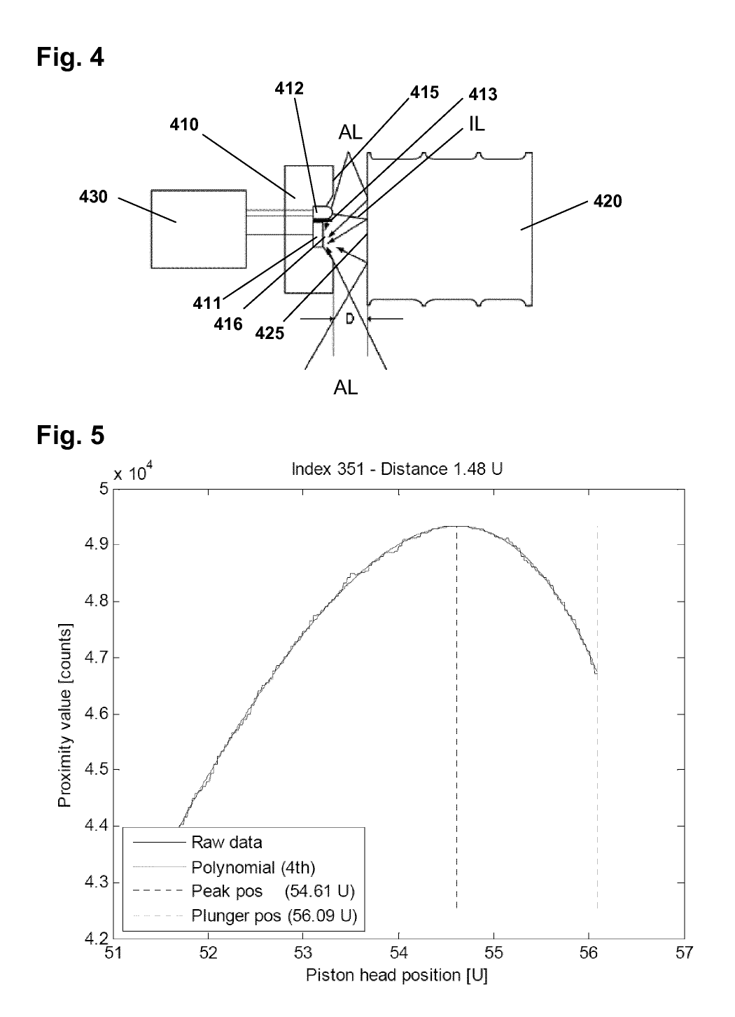

In a second exemplary embodiment engagement is detected using an optical sensor system arranged at the distal end of the piston rod. Referring to FIG. 4 a schematic representation of components of a drug delivery sensor system is shown. The system comprises a piston drive member in the form of a piston rod washer 410 mounted on a piston rod (not shown) and driven by an electronically controlled motorized mechanism (not shown), a piston 420 mounted in a cartridge (not shown), and a controller 430. The piston rod washer comprises a distal surface 415 adapted to engage a proximal surface 425 on the piston, the distal surface being provided with a central cavity 416 in which a light sensor 411 and a light source 412 in the form of a IR LED are arranged next to each other with a barrier member 413 being mounted there between. The IR LED is arranged to direct IR light IL towards the proximal surface of the piston and the light sensor is arranged to detect the reflected light therefrom, the barrier preventing direct light from the IR LED to reach the sensor. Also ambient light AL may reach the sensor, however, the IR component of the AL is small compared to the IR component from the IR LED.

Referring to FIG. 5 data from a test set-up is shown in which the axial position is shown as units of insulin based on a test set-up with a 3 ml standard Novo Nordisk Penfill® cartridge. The figure shows the raw output of the proximity sensor and a fitted polynomial (the smooth curve). Contact of the piston is at the dotted line. The algorithm needs to fit the curve and find the peak, thus some of the readings in the region of decreasing values is needed as input to the algorithm before the result is ready and the piston position can be estimated before contact, the accuracy gets better the more of the signal that can be used. In the FIG. 5 example piston engagement was found to take place 1.48 U after curve peak. The sensor reading values at piston engagement will depend upon the actual sensor design. It appears that a motorized drug delivery device with on-the-fly piston engagement detection can be provided using a distally-mounted piston rod optical sensor, however, the optical sensor system may be less robust than the above-described force sensor system as for example variations for the piston proximal surface, e.g. due to dirt, would influence measurements.

As detection of piston engagement in the above-described sensor embodiments is based on detection of a curve peak, piston rod movement has to start with a certain air gap between the piston rod and the piston. This may be realized as described above by retracting the piston rod after an expelling event. Alternatively an expelling event may be initialized by retracting the piston rod a certain amount.

Turning to embodiments of the present invention in accordance with the second general aspect, the above-described drive arrangement for a motorized drug delivery device is adapted to cope with the issue of freezing drug in the cartridge, e.g. an insulin formulation.

More specifically, when a drug delivery device with a loaded cartridge is placed in a refrigerator the drug, e.g. insulin, may accidentally freeze if the device is placed in close proximity to the evaporator or the cold air outlet. The drug thereby expands and the piston rod arranged in contact with the cartridge piston is pushed back with a high force which may seriously harm the device drive mechanism.

Addressing this issue, the present invention provides a motorized drug delivery device adapted to protect the device drive mechanism from damage when the cartridge piston is moved proximally due to freezing and thereby expanding drug.

In a first exemplary embodiment a sensor is provided to generate an output indicative of a proximally directed force exerted on the piston rod. The sensor may be a force sensor arranged at the distal-most portion of the piston rod as described above feeding signals to a processor. The force sensor may be used for other purposes as well, e.g. to detect when the piston rod is moved into contact with the piston after cartridge change. When a force above a given minimum is detected this is considered indicative of freezing drug, this resulting in the processor controlling the drive mechanism to move the piston rod a certain amount in the proximal direction. To avoid false detection of a “freeze event”, e.g. during needle exchange or if the device is dropped on the floor, the processor may be adapted to discriminate between such situations. For example, a freeze event may be detected when a given near-constant force is detected over a given period of time.

In a second exemplary embodiment a temperature sensor is provided to generate an output indicative of a temperature in the cartridge. The sensor may be arranged at a distance from the cartridge, e.g. as part of the main electronic circuitry to provide a simple and cost-effective design, the detected temperature being correlated with an assumed actual temperature in the cartridge by e.g. experimental data. When a temperature below a given value is detected this is then considered indicative of freezing drug (or the drug being at risk of freezing), this resulting in the processor controlling the drive mechanism to move the piston rod a certain amount in the proximal direction.

Indeed, when the piston rod has been moved out of engagement with the cartridge piston, it has to be moved into engagement with the piston again in conjunction with the device being operated to expel a dose of drug. In order to prevent the user from using the device with frozen drug, a warning may be given when the device is turned on after detection of a freeze event, this indicating that the user should check the drug condition. Alternatively, the device may be adapted to be in-operatable until a cartridge exchange procedure has been performed.

In a third exemplary embodiment the device is not adapted to detect actual conditions, however, after each expelling event the device is turned into a “protected state” in which the expelling mechanism cannot be damaged by a proximally-moving piston. In a specific embodiment the piston rod is moved proximally a given distance after each expelling event (or other event in which the piston rod has been in engagement with the cartridge piston), this providing a gap between the piston rod and piston sufficient to accommodate the piston being forced proximally during a potential freezing event. As an electronically controlled drug delivery device normally will keep track of the amount of drug left in a loaded cartridge, the distance the piston rod is moved proximally may be correlated with the actual amount of drug in the cartridge, i.e. the more drug in the cartridge the more proximally the piston rod is moved, this reducing energy consumption. The piston rod may be moved back into contact with the piston e.g. when the user turn on the device, when the user start to set a dose or just prior to an out-dosing event. In an alternative embodiment a coupling is provided between the piston rod and the drive mechanism, the coupling having a first operational state in which operation of the drive mechanism moves the piston rod distally, and a second protected state in which the piston rod can be moved proximally by the piston during a freeze event.

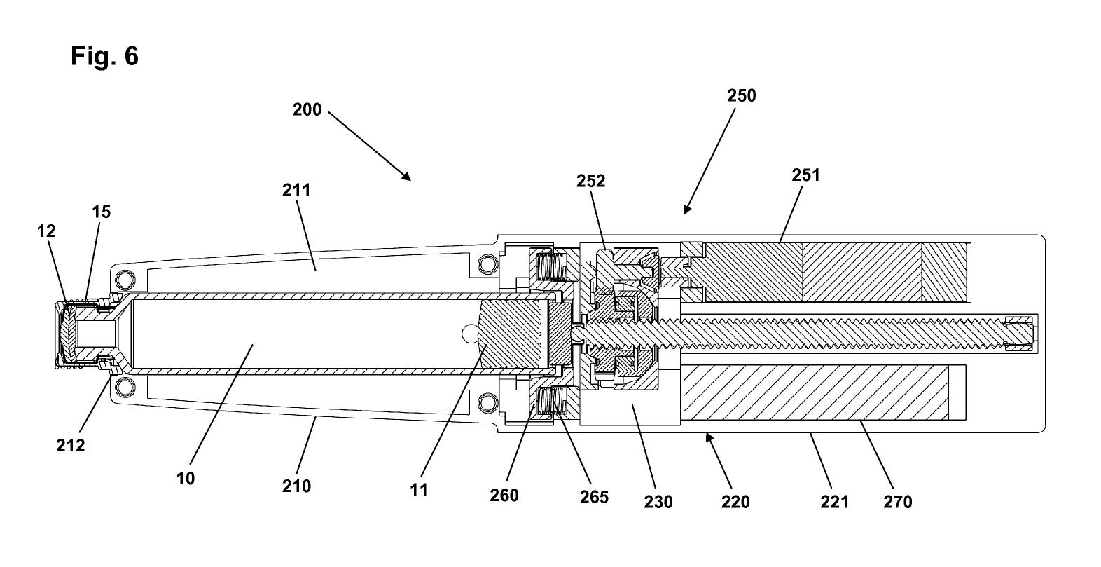

Turning to FIG. 6 a first embodiment 200 of a drug delivery device suitable as a platform for embodiments of the present invention will be described. More specifically, the device comprises a cap part (not shown) and a main part having a proximal body or drive assembly portion 220 with a housing 221 in which a motorized drug expelling assembly 250, electronic controller circuitry 270 and an electric power source are arranged, and a distal cartridge holder portion 210 with a compartment 211 in which a drug-filled cartridge 10 is arranged and retained in place. The cartridge comprises a generally cylindrical main portion with an axially displaceable piston 11 and a distal outlet portion 12 comprising a needle-penetrable septum. The cartridge is further provided with distal coupling means in the form of a needle hub mount 15 having, in the shown example, an external thread adapted to engage an inner thread of a corresponding hub of a needle assembly. The cartridge may for example contain an insulin, a GLP- 1 or a growth hormone formulation. The device further comprises dose setting means allowing a user to set a dose of drug to be expelled as well as a display showing the set dose, e.g. as shown in FIG. 1.

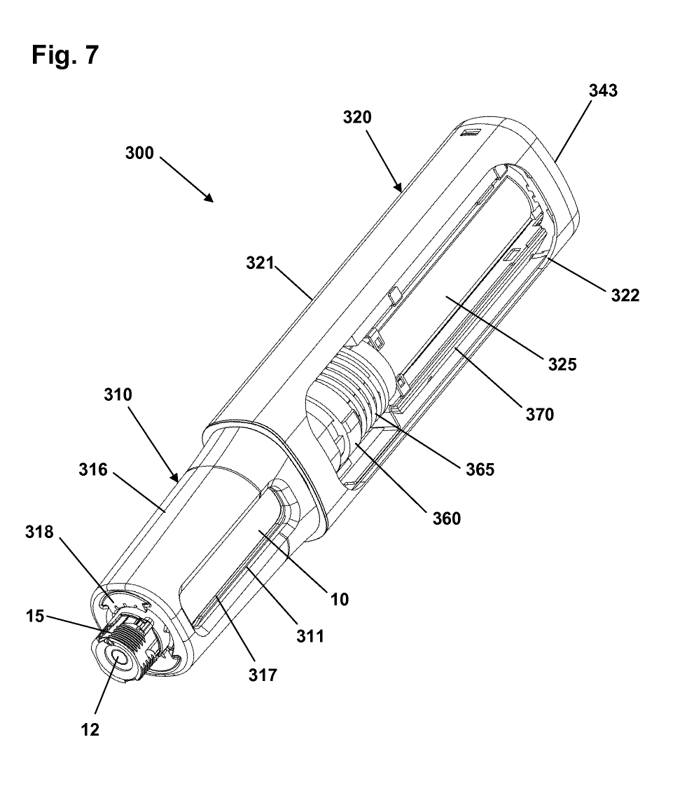

In the shown embodiment the device is designed to be loaded by the user with a new cartridge through a distal receiving opening 212 in the cartridge holder assembly, the cartridge holder comprising closure means (not shown) operatable by a user between an open position in which a cartridge can be inserted respectively removed, and a closed position in which an inserted cartridge is held in place. The closure means may be of the same type as described with respect to FIG. 7 below. In order to axially position the cartridge, the device comprises a seat member 260 adapted to receive the proximal end of the cartridge, the seat member being biased in the proximal direction by springs 265 thereby forcing the cartridge into contact with the closure means.

Turning to FIG. 7 a second embodiment 300 of a drug delivery device suitable as a platform for embodiments of the present invention will be described. More specifically, the device comprises a cap part (not shown) and a main part having a proximal body or drive assembly portion 320 with a housing 321 in which a drug expelling mechanism and associated electronics 370 are arranged, and a distal cartridge holder assembly 310 forming a compartment in which a drug-filled transparent cartridge 10 can be arranged and retained in place, the cartridge holder assembly comprising a pair of opposed inspection openings 311. The housing comprises an opening 322 adapted to receive a display frame member (not shown) in which a LCD as well as user input keys are mounted, e.g. as shown in FIG. 1. With the frame member removed, it can be seen that the device comprises a generally tubular chassis member 325, in which a generally cylindrical expelling assembly is mounted (see below). The device further comprises a control assembly 370, a bias assembly comprising a bias member 360 and a spring 365, and a proximal release button 343. A pair of dose setting input keys (not shown) serves to manually set a desired dose of drug shown in the LCD and which can then be expelled when the release button 90 is actuated. The device is designed to be loaded by the user with a new cartridge through a distal receiving opening in the cartridge holder assembly.

The cartridge 10 comprises a cylindrical body portion, a distal outlet portion 12 with a distal needle-penetrable septum, and an axially displaceable piston having a proximal surface allowing a piston driver forming part of the expelling mechanism (see below) to engage the piston. The cartridge may for example contain an insulin, a GLP- 1 or a growth hormone formulation. The cartridge is provided with distal coupling means in the form of a needle hub mount 15 having, in the shown example, combined thread and bayonet coupling means, each being adapted to engage an inner thread or bayonet coupling means of a corresponding hub of a needle assembly. The shown exemplary hub mount further comprises a circumferential flange with a number of distally facing pointed projections serving as a coupling means for the cartridge holder assembly as will be described in more detail below. A hub mount of the shown type is described in U.S. Pat. No. 5,693,027. Alternatively the needle hub mount may be formed as part of the cartridge holder, e.g. in the form of a “split” hub mount having two parts arranged on each side of the gripping shoulders.

As shown, the cartridge holder assembly 310 has the same general appearance as a traditional cartridge holder which is detachably coupled to the housing by e.g. a threaded coupling or a bayonet coupling and into which a new cartridge can be received as well as removed through a proximal opening, i.e. it comprises no additional user operated release or locking means. Instead, what appears merely to be the cartridge holder per se is in fact user operated coupling means in the form of an outer rotatable tubular actuation sleeve 316 operated by the user to control movement of cartridge holding means in the form of an inner cartridge holder member 317 to thereby open and close gripping shoulders 318 configured to grip and hold a cartridge. More specifically, each gripping shoulder is provided with a plurality of gripping teeth spaced circumferentially to provide a plurality of gaps, each tooth having a triangular configuration with a proximally oriented pointed end, thereby creating a plurality of gaps having a distally oriented pointed configuration, this allowing the above-described distally facing pointed projections on the cartridge to be received between the teeth to thereby serve as a gripping means when the cartridge holding means has been moved into engagement with the cartridge. In this way an easy-to-use front loaded drug delivery device is provided which appears as a traditional rear loaded device and which is also actuated by rotational movement to mount and remove a cartridge, the resemblance providing for ease of acceptance and adaptation among users accustomed to traditional types of rear loaded drug delivery devices.

When it is time to mount a new cartridge the outer tube member 316 is rotated e.g. 90 degrees by which action the gripping shoulders 318 are moved distally and slightly outwards, this allowing the mounted cartridge to be removed. For ease of operation the cartridge may be moved distally a certain distance as the shoulders are moved, e.g. by engagement with arms forming the gripping shoulders and/or by additional spring means providing a biasing distally directed force (see below). Depending on the design of the locking and actuation mechanism the gripping shoulders may be able to be left in the open position or they may be retracted automatically as the outer tube member is rotated backwards by return spring means. Whether or not a spring is provided the cartridge holder may be provided with locking means allowing the outer tube member to be securely parked in either the open or closed position, e.g. by a rotational snap lock. When a new cartridge is inserted the drive expelling means has to be in a state allowing a new cartridge with a proximally positioned piston to be inserted. An exemplary embodiment providing this functionality will be described below.

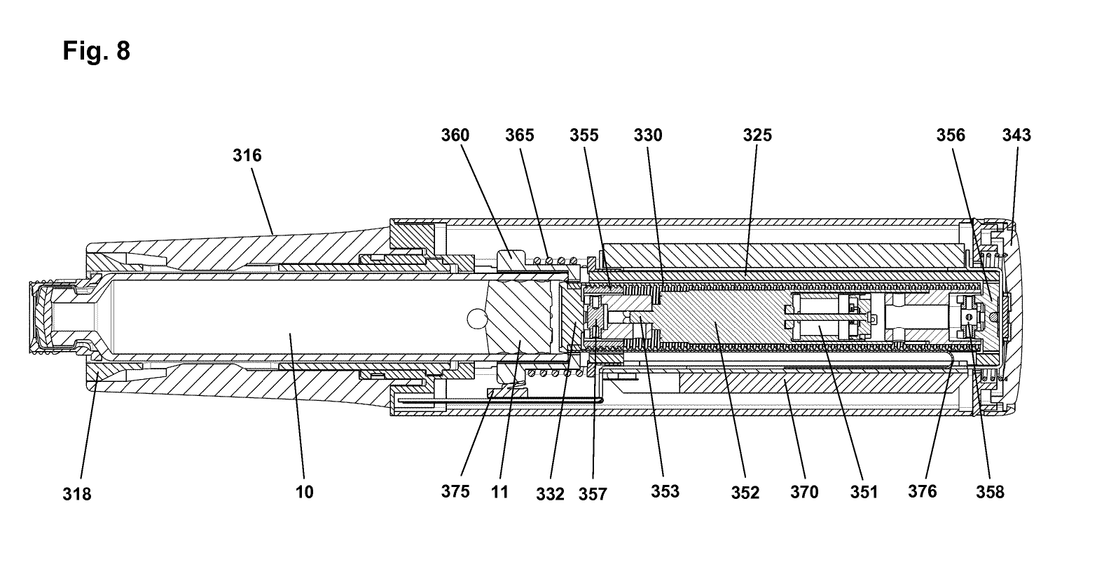

Turning to FIG. 8 a cross-sectional view of the drug delivery device 300 of FIG. 7 is shown with a mounted cartridge 10 and with the piston tube 330 (see below) in a fully retracted position. More specifically, the actuation sleeve 316 has been rotated to its operational position and the cartridge holder gripping shoulders 318 have been retracted to their closed position thereby retracting the cartridge to its fully inserted position, thereby also moving the bias member 360 proximally against the bias of the spring 365. In the shown embodiment a cartridge switch 375 is hereby being actuated, this providing a signal to the device controller that two actions can be assumed to have taken place: (i) a cartridge has been inserted and (ii) the cartridge holder has been closed, this initiating that the drive head is moved distally into contact with the cartridge piston. In the shown embodiment it is contemplated that detection of contact between the drive head and the piston is detected by electronic sensor means arranged in the drive head, e.g. using proximity detection as disclosed in WO 2013/144152.

FIG. 8 also shows the expelling assembly in greater detail. More specifically, the expelling assembly is in the form of a motor-in-piston assembly comprising an interior motor and gearbox drive assembly mounted axially and rotationally locked to the proximal end of the chassis, and an outer axially displaceable piston tube 330 with a distal drive head 332 adapted to engage the piston 11 of a loaded cartridge, the piston tube comprising a number of guide projections adapted to non-rotationally engage corresponding guide means of the chassis.

The motor-gear drive assembly comprises a tubular main portion 310 composed of a proximal motor assembly 351 and a distal gearbox assembly 352 having a rotatable drive shaft 353 defining a z-axis of rotation. The assembly further comprises a distal cylindrical drive member 355 having an outer thread adapted to be arranged in engagement with the piston drive tube inner thread. At the proximal end a disc-formed chassis connector 356 is arranged. In the shown embodiment the drive assembly is provided with flexible joints in the form of a distal universal joint 357 arranged between the drive shaft and the drive member and a proximal universal joint 358 arranged between the motor assembly proximal portion and the chassis tube proximal portion. A corresponding drive assembly is described in greater detail in patent application EP 14166859.0, which is hereby incorporated by reference.

A number of further details can be seen in FIG. 8. The release button 343 is received in the housings proximal opening with a spring providing a proximally directed biasing force on the button. A flexible ribbon 376 with a plurality of conductors is arranged with a U-bend between the electronics portion 370 and the sensors (not shown) arranged in the piston head, this allowing the piston tube and piston head to travel axially with the U-bend moving correspondingly.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drug delivery device, comprising:
a drug-filled cartridge or structure for receiving a drug-filled cartridge in a loaded position, the cartridge comprising an outlet and an axially displaceable piston,
drug expelling structure comprising:
structure means for setting a dose corresponding to a desired dose amount,
a drive member adapted to engage and axially move the piston to thereby expel an amount of drug from a loaded cartridge through the outlet corresponding to the set dose amount, and
a motor for moving the drive member from an initial position, through a start-of-dose position, to an end-of-dose position,
position detection structure adapted to generate output data indicative of the drive member's position relative to the piston of a loaded cartridge,
an electronic controller adapted to:
operate the motor to move the drive member from the initial position,
based on output data from the position detection structure determine that the drive member is in engagement with the cartridge piston corresponding to the start-of-dose position, and
operate the motor to move the drive member from the start-of-dose position to the an end-of-dose position corresponding to the set dose,
wherein the motor is operated continuously as the drive member is moved from the initial to the end-of-dose position.

2. A drug delivery device as in claim 1, wherein the electronic controller is adapted to determine the start-of-dose position based on a time-shifted calculation.

3. A drug delivery device as in claim 2, wherein the start-of-dose position is determined after the drive member has engaged the cartridge piston.

4. A drug delivery device as in claim 3, wherein the position detection structure comprises a force sensor arranged at least partly in the drive member.

5. A drug delivery device as in claim 2, wherein the start-of-dose position is determined before the drive member engages the cartridge piston.

6. A drug delivery device as in claim 5, wherein the position detection structure comprises a proximity sensor arranged at least partly in the drive member.

7. A drug delivery device as in claim 1, wherein the motor is operated to move the drive member proximally a distance after the drive member has reached the end-of-dose position.

8. A drug delivery device as in claim 1, wherein the motor is operated to move the drive member proximally a distance from the initial position before the drive member is moved continuously from the initial to the end-of-dose position.

9. A drug delivery device as in claim 1, wherein the motor is operated to move the drive member at a non-constant speed.

10. A drug delivery device as in claim 1, wherein the electronic controller is adapted to control the motor to move the drive member in a proximal direction to thereby allow the content of a drug-filled cartridge to expand.

11. A drug delivery device as in claim 10, further comprising a temperature sensor adapted to measure a temperature indicative of the temperature in a loaded cartridge,
wherein the controller, based on data from the temperature sensor, is adapted to move the drive member proximally when a given temperature is measured.

12. A method of operating a drug delivery device, comprising the steps of:
providing a drug delivery device comprising:
a drug-filled cartridge comprising an outlet and an axially displaceable piston,
drug expelling structure comprising:
structure for setting a dose corresponding to a desired dose amount,
a drive member adapted to engage and axially move the piston to thereby expel an amount of drug from a loaded cartridge through the outlet corresponding to the set dose amount, and
a motor for moving the drive member from an initial position, through a start-of-dose position, to an end-of-dose position,
position detection structure adapted to generate output data indicative of the drive member's position relative to the piston of a loaded cartridge, and
an electronic controller,
operating the motor to move the drive member from the initial position,
based on output data from the position detection structure determining that the drive member is in engagement with the cartridge piston corresponding to the start-of-dose position, and
operating the motor to move the drive member from the start-of-dose position to the an end-of-dose position corresponding to the set dose,
wherein the motor is operated continuously as the drive member is moved from the initial to the end-of-dose position.

13. A method of operating a drug delivery device as in claim 12, wherein determining the start-of-dose position is based on a time-shifted calculation.

14. A method of operating a drug delivery device as in claim 13, wherein the start-of-dose position is determined after the drive member has engaged the cartridge piston.

15. A method of operating a drug delivery device as in claim 13, wherein the start-of-dose position is determined before the drive member has engaged the cartridge piston.
